(19) United States
(12) Patent Application Publication
(10) Pub. No.: US 2005/0196431 A1
(43) Pub. Date: Sep. 8, 2005

(54) Adhesive applicator tip with a polymerization initiator, polymerization rate modifier, and/or bioactive material

(76) Inventors: Upvan Narang, Raleigh, NC (US); William S.C. Nicholson, Raleigh, NC (US); Daniel L. Hedgpeth, Raleigh, NC (US); Gabriel N. Szabo, Raleigh, NC (US); Ibraheem T. Badejo, Morrisville, NC (US); Joe B. Barefoot, Raleigh, NC (US); William M. Cotter, Raleigh, NC (US); Keith R. D'Alessio, Cary, NC (US); Anthony Sherbondy, Raleigh, NC (US)

Correspondence Address: HUTCHISON & MASON PLLC, PO BOX 31686, RALEIGH, NC, 27612, US

(21) Appl. No.: 11/119,992

(22) Filed: May 2, 2005

Related U.S. Application Data
(63) Continuation of application No. 09/430,177, filed on Oct. 29, 1999.
Application No. 09/430,177 is a continuation-in-part of application No. 09/069,979, filed on Apr. 30, 1998, now abandoned.

(51) Int. Cl. A61M 35/00; A61L 15/16

(52) U.S. Cl. 424/444; 604/1

(57) ABSTRACT

An applicator tip for an applicator for applying a polymerizable monomeric adhesive composition can include a bioactive material, a flavorant, a polymerization initiator, and/or a polymerization rate modifier.

This application is a continuation-in-part of U.S. patent application Ser. No. 09/069,979, filed Apr. 30, 1998, the entire disclosure of which is incorporated herein by reference.

### BACKGROUND OF THE INVENTION

1. Field of the Invention

This invention relates to applicators for applying biomedical adhesives and sealants, methods of making them, and methods of applying such adhesives and sealants. More particularly, this invention relates to methods of applying a bioactive agent, polymerization rate modifier, and/or polymerization initiator to an applicator tip; applicators and applicator tips produced by such methods; and methods of using the applicators in medical, surgical, and other topical applications.

2. Description of Related Art

Products in primary use for wound closure are surgical sutures and staples. Sutures are recognized to provide adequate wound support. However, sutures cause additional trauma to the wound site (by reason of the need for the needle and suture to pass through tissue and the need to anesthetize the wound area via needle puncture) and are time-consuming to place, and, at skin level, can cause unattractive wound closure marks. Surgical staples have been developed to speed wound apposition and provide improved cosmetic results. However, surgical staples also impose additional wound trauma and require the use of ancillary and often expensive devices for positioning and applying the staples. Both sutures and staples are especially problematic in pediatric cases where the patient may have a strong fear response and refuse to cooperate with their placement, and in geriatric cases where the skin tissue is weaker and prone to tearing.

As an alternate to surgical sutures and staples, adhesives have been proposed for use in wound closure. Similarly, adhesives have been proposed for use in wound covering and protection in such topical applications as surface lacerations, abrasions, stomatitis, and other open surface wounds. One group of such adhesives is the monomeric forms of α-cyanoacrylates.

Typically, for wound closure, the cyanoacrylate surgical adhesive is applied to one or both surfaces of a wound or incision, including the internal portions of the wound, with any excess adhesive being quickly removed from the bonding surfaces. Subsequently, the edges of the wound are held together until they adhere. For example, see U.S. Pat. No. 3,559,652 to Coover, Jr. et al. An additional method of application of the cyanoacrylate surgical adhesive to wounds or incisions involves the formation of a bridge over the wound site. As described in U.S. Pat. No. 3,667,472 to Halpern, incised tissues are held together and maintained in fixed relationship until a cyanoacrylate adhesive has been applied over the incision and allowed the necessary time to develop a bond.

A method for application of a topical cyanoacrylate tissue adhesive is disclosed in product literature accompanying Histoacryl® adhesive, which is commercially available from B. Braun Melsungen AG of Germany. The manufacturer recommends use of this adhesive only for closure of minor skin wounds and not for internal use. Moreover the manufacturer recommends that the adhesive be used sparingly or in thin films because thick films do not increase the film strength and can lead to necrosis of surrounding tissue due to thermogenic polymerization of the cyanoacrylate adhesive.

Typically, when used in medical applications, cyanoacrylate adhesives are applied in monomeric form to the surfaces to be joined, sealed, or otherwise treated. Typically, in situ anionic polymerization of the monomer occurs, giving rise to the desired adhesive bond or covering. In these instances, moisture and/or proteins naturally present in the treated tissues initiate polymerization of the adhesives. However, as is the case with Histoacryl® adhesive, polymerization can proceed rapidly, with the generation of high levels of heat, which often damage the tissues at or near the site of application.

In an effort to overcome this type of tissue damage, the tissue can first be dried, for example by sponging, to remove essentially all tissue fluids from the site. In this method, there is essentially no water to initiate polymerization. Therefore, polymerization proceeds relatively slowly, often taking greater than 150 seconds, for example. This system, while effective, does not provide a high level of convenience for the user due to the extended time often required for polymerization.

In view of shortcomings associated with the methods disclosed above, an effort has been made to control the rate at which polymerization occurs such that polymerization will occur rapidly enough to be convenient for the user, but not so rapidly that tissue damage occurs due to the polymerization reaction. To control the rate at which the adhesives polymerize (and to improve the shelf life), additives have been included in the monomer adhesive compositions. For example, cyanoacrylate polymerization inhibitors or stabilizers including Lewis acids, such as sulfur dioxide, nitric oxide, boron trifluoride, and other acidic substances, including hydroquinone monomethyl ether, hydroquinone, nitrohydroquinone, catechol, and hydroquinone monoethyl ether have been used. Such inhibitors are disclosed in, for example, U.S. Pat. No. 3,559,652 to Banitt, the subject matter of which is incorporated herein by reference. The addition of these inhibitors and stabilizers inhibits premature polymerization of the monomer and slows down the rate of polymerization once the composition is in contact with the tissue to be treated.

Another method for inhibiting polymerization of monomeric adhesives is disclosed in U.S. Pat. No. 4,291,131 to McIntire et al. McIntire et al. disclose a nozzle for use on containers for holding cyanoacrylate adhesives so that the cyanoacrylate compositions do not begin to polymerize on exposure to moisture in the air. The nozzle comprises a moldable material having an organic acid dispersed therein that inhibits the polymerization of cyanoacrylates while in the nozzle. The organic acids are incorporated into the moldable material prior to extrusion forming of the nozzle.

Although it is known to add polymerization inhibitors and stabilizers to cyanoacrylate compositions to increase stability and shelf life of the compositions, the addition of polymerization initiators or accelerators to the cyanoacrylate compositions is not widely performed. As discussed above, polymerization typically occurs in situ without the need for an external initiator or accelerator. In the situations where an initiator or accelerator is added to the composition, such as when tissue fluids have been removed from the application site, the initiator or accelerator is not added until immediately prior to application of the adhesive. For example, U.S. Pat. No. 4,042,442 to Dombroski et al. discloses the addition of a polymerization initiator (either caffeine or theobromine) to a cyanoacrylate adhesive composition. The caffeine or theobromine is added to the adhesive composition in one of two ways. In the first way, the caffeine or theobromine can be mixed with the cyanoacrylate adhesive composition by stirring just prior to application of the adhesive to the substrates to be joined. In the second way, the caffeine or theobromine is dissolved in a volatile solvent, applied to the surfaces to be joined, the volatile solvent is allowed to evaporate, and then the cyanoacrylate adhesive composition is applied to the surfaces of the substrates to be joined. Both of these methods, while effective, are inconvenient for the user because two separate solutions or two separate applications are required.

In an effort to address this inconvenience and lack of control over the polymerization process, commonly assigned U.S. Pat. No. 5,928,611 (corresponding to earlier-published PCT Application No. WO 96/40797), the disclosure of which is hereby incorporated in its entirety, discloses the incorporation of a polymerization initiator or polymerization rate modifier on an applicator tip. Incorporation of the initiator or the rate modifier into the applicator tip provides convenience because only a single composition is required, and allows a level of control over the polymerization rate that cannot be achieved through reliance on polymerization initiators or rate modifiers naturally present at the wound site (such as water).

The polymerization initiators and/or rate modifiers are incorporated into the applicator tip by spraying, dipping, or brushing the applicator tip with a solvent (also referred to herein as a liquid medium) containing the initiator and/or rate modifier. Low boiling point solvents (such as acetone and ethanol, or mixtures thereof) are used to apply the initiator and/or rate modifier.

The applicator tips disclosed in this commonly assigned patent effectively and conveniently permit mixing of a cyanoacrylate composition with a polymerization initiator or a polymerization rate modifier during dispensing. The polymerization reaction that ensues, however, can be highly exothermic, and, like other methods currently in use, can cause tissue damage at the site of application due to excessive heat generation during polymerization.

In addition to adding polymerization inhibitors, stabilizers, and initiators to monomeric cyanoacrylate compositions, it is also known to add bioactive materials to these adhesive compositions. Often, these bioactive materials are medicaments which are added to the adhesive compositions to aid in the healing process when the cyanoacrylate adhesives are used to close wounds. For example, U.S. Pat. No. 5,684,042 to Greff et al. discloses a cyanoacrylate composition comprising an antimicrobially-effective amount of an iodine-containing antimicrobial agent. The iodine-containing antimicrobial agent is dispersible in the cyanoacrylate composition and does not cause premature polymerization of the cyanoacrylate adhesive (i.e., does not initiate polymerization).

Additionally, U.S. Pat. Nos. 5,514,371 and 5,624,669 to Leung, et al. disclose the addition of a therapeutic agent in a cyanoacrylate composition. The cyanoacrylate adhesive forms a matrix for the therapeutic agent, with the therapeutic agent being released in vivo over time from the matrix during biodegradation of the polymer.

U.S. Pat. No. 4,940,579 to Randen discloses a composition comprising a medicament and a cyanoacrylate adhesive. The composition is used to deliver medicaments to non-mucosal areas of mammal bodies.

U.S. Pat. No. 5,254,132 to Barley et al. discloses the use of cyanoacrylate adhesives in conjunction with antibiotics. The antibiotics are added to the cyanoacrylate compositions and stored in a sterile applicator for use in a single-dose application. The composition is maintained in a sealed container to avoid polymerization prior to application; therefore, the antibiotic does not initiate or accelerate polymerization of the adhesive composition.

U.S. Pat. No. 5,866,106 to Papay discloses the addition of vitamins and minerals in a cyanoacrylate composition. The cyanoacrylate adhesive composition is disclosed as useful for an adhesive for bonding nail tips, and for forming a nail polish product.

Commonly assigned U.S. patent application Ser. No. 09/343,914, filed Jun. 30, 1999, discloses monomeric adhesive composition comprising a polymerizable 1,1-disubstituted ethylene monomer and a flavoring additive, and methods of making and using such a composition. In these compositions, the flavoring additive is mixed directly with the monomeric adhesive.

While all of these methods include combining cyanoacrylate adhesives with bioactive materials, the disclosed methods are inconvenient for applying adhesive compositions because multiple solutions and/or applicators are required in order to mix the initiator and adhesive composition or fail to provide a way of controlling the rate at which polymerization proceeds. Furthermore, the selection of bioactive materials has generally been limited by the desire to avoid interaction between the adhesives and the bioactive materials.

### SUMMARY OF THE INVENTION

It has been discovered that the use of methanol, alone or as a component of a mixture of low boiling point solvents, to apply a material (such as a polymerization and/or cross-linking initiator or rate modifier) to an applicator tip used to dispense monomer-containing adhesive compositions, provides an unexpectedly superior distribution profile of the material on, and within, the applicator tip. The superior distribution profile allows a reduction in polymerization time of the dispensed monomeric adhesive while avoiding tissue damage due to the highly exothermic polymerization reaction. It has also been discovered that bioactive materials and/or flavorants, which can be polymerization initiators and/or rate modifiers as well, can be applied to applicator tips, providing improved convenience when treating a tissue.

In one aspect, the present invention provides a method of applying at least one material to an applicator tip used to dispense liquid compositions. In embodiments, the material may be applied to the applicator tip using a solvent comprising methanol and further comprising another low boiling point solvent, such as a low molecular weight ketone or alcohol, or a mixture thereof. As used herein, low molecular weight ketones and alcohols are those which have three or fewer carbon atoms in their main chain. In preferred embodiments, the solvent consists essentially or entirely of methanol.

In other aspects of the present invention, the material is incorporated into the applicator tip during the manufacturing process of the applicator tip. In embodiments, a desired distribution profile of the material on, and/or within, the applicator tip can be achieved without the need for an extra step of applying the material to an already formed applicator tip.

In embodiments, the material is applied to an applicator tip such that the material is present on the tip in a gradient or anisotropically (i.e., in a pattern that is not identical in all directions within the tip). Preferably, the material is present on the tip in a gradient, where there is a greater amount of the material at the distal end of the tip (the end where the liquid composition exits the applicator tip during dispensing) as compared to the proximal end (the end where the liquid composition enters the applicator tip during dispensing).

In embodiments, the material is an initiator and/or a rate modifier for polymerization and/or cross-linking of a polymerizable monomer. As used herein, a polymerization initiator is any material that causes a monomer composition applied to a substantially dry tissue (i.e., substantially in the absence of plasma or like tissue fluids) to polymerize in less than 300 seconds at ambient temperature, for example, at approximately 21-25° C. Preferably, the initiator causes the monomer composition to polymerize in less than 150 seconds at ambient temperature, more preferably within 130 seconds. As used herein, a polymerization rate modifier is any material that changes the rate at which a polymerizable monomer would polymerize in the absence of that material. Preferably, the rate modifier accelerates the rate of the polymerization reaction.

In embodiments, the initiator or rate modifier is an accelerator or catalyst. In embodiments, the initiator and/or rate modifier is bioactive. In other embodiments, the material applied to the tip is bioactive or a flavorant, but not an initiator or rate modifier for polymerization and/or cross-linking of the polymerizable monomer.

The present invention also provides a method of using an applicator tip containing a polymerization and/or cross-linking initiator, a polymerization and/or cross-linking rate modifier, and/or a bioactive material and/or a flavorant to apply a monomeric composition to a desired site, such as a wound, a surgical site, or any other topical or deep tissue site. In embodiments, the method is used to treat wounds or to treat or protect topical sites, such as areas of skin prone to wounding.

The present invention also provides a method of delivering a bioactive material to a tissue. As used herein, tissue includes any tissue of a human or animal, such as skin, mucous membranes, oral/nasal tissues, gastrointestinal tissues, organ tissues, tumors, non-keratinous tissues, etc.

The present invention further provides an applicator tip that has a polymerization or cross-linking initiator, a polymerization and/or cross-linking rate modifier, and/or a bioactive material and/or a flavorant on or in it.

Applicator tips according to the present invention provide several advantages, including the ability to:
a) control the molecular weight distribution of the polymerized or cross-linked adhesive;
b) control the setting time of the polymerized or cross-linked adhesive;
c) provide precision and convenience in applying the adhesive to a tissue;
d) extend the shelf life of the monomer;
e) reduce the amount of unreacted monomer at the completion of the polymerization reaction, thus avoiding associated monomer odors after polymerization;
f) control the flow properties of applied cyanoacrylate adhesives;
g) provide a bioactive material and/or flavorant to a wound site while simultaneously providing wound closure, protection, and/or coverage; and/or
h) combinations thereof.

### BRIEF DESCRIPTION OF THE DRAWINGS

FIG. 1 depicts an applicator of the invention, showing the distribution of a polymerization rate modifier or a polymerization initiator in an applicator tip applied with methanol.

FIG. 2 shows a cross section of the applicator of FIG. 1 along the A-A line.

FIG. 3 depicts an applicator showing the distribution of a polymerization rate modifier or a polymerization initiator on an applicator tip applied with acetone.

FIG. 4 shows a cross section of the applicator of FIG. 3 along the A-A line.

FIG. 5 shows the polymerization. temperatures of a 2-octyl cyanoacrylate composition dispensed through applicator tips having an initiator applied with methanol and with acetone.

FIG. 6 shows the relationship between setting time (time required for polymerization) of a 2-octyl cyanoacrylate composition and concentration of initiator. The figure also shows the time required for polymerization of a 2-octyl cyanoacrylate composition dispensed through tips having an initiator applied with methanol and with acetone.

### DETAILED DESCRIPTION OF PREFERRED EMBODIMENTS

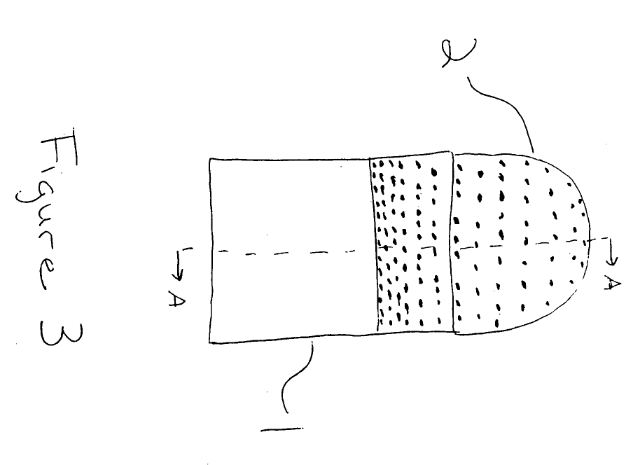

As mentioned above, commonly assigned U.S. Pat. No. 5,928,611, the disclosure of which is hereby incorporated in its entirety, discloses the use of low boiling point solvents, such as acetone, ethanol, or mixtures thereof, to apply a polymerization or cross-linking initiator or rate modifier to an applicator tip. Cyanoacrylate adhesive compositions applied through such tips can show rapid polymerization, with concomitant production of heat. If the compositions are applied to living tissues, this might, in some instances, cause damage to the tissues and necrosis of underlying and/or adjacent living matter. Analysis of the tips produced by the methods of this patent shows that the use of acetone to apply the initiator or rate modifier to the tip results in distribution of the material primarily on the outside, or near the exterior surface, of the tip as well as at the proximal, or bottom, portion. Such a distribution is shown in FIG. 3.

To improve the polymerization characteristics of polymerizable monomer compositions delivered through applicator tips having a polymerization initiator and/or a polymerization rate modifier disposed thereon, the solvent used to apply the initiator or rate modifier was varied. It was discovered that the use of methanol to apply the initiator or rate modifier to an applicator tip provides unexpectedly superior polymerization characteristics to a polymerizable monomer composition in comparison to compositions applied through tips having an initiator and/or rate modifier applied using acetone. Polymerizable cyanoacrylate monomer compositions dispensed through such tips typically generate much less heat than the same compositions dispensed through tips prepared using acetone.

It can be shown, using thermal analysis techniques such as differential scanning calorimitry, that monomer compositions applied through such tips generate levels of heat that can be damaging to tissues. For example, as shown in FIG. 5, a composition comprising 2-octyl cyanoacrylate dispensed through an applicator tip having an initiator (benzalkonium chloride) applied with acetone can generate enough heat at 200 seconds after initiation of polymerization to raise the temperature of the composition to approximately 80° C. However, the same composition dispensed through a tip having the same initiator applied using methanol shows a much lower level of heat generation (approximately 40° C).

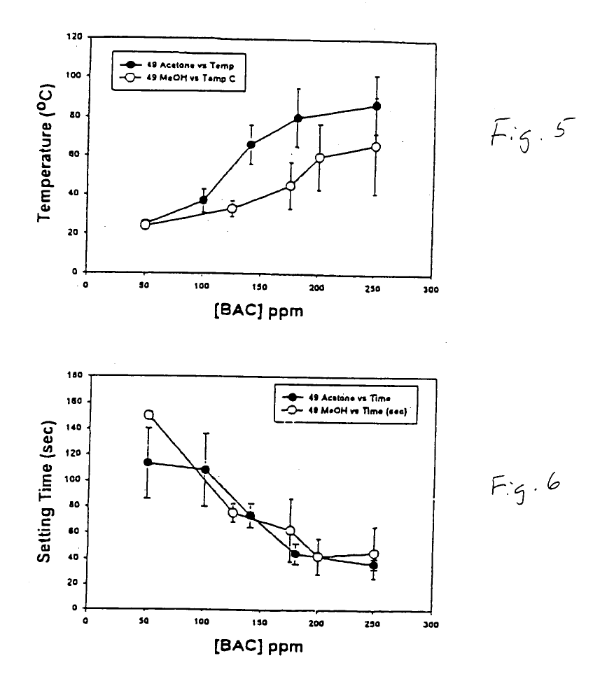

Furthermore, as shown in FIG. 6, the setting time, or the amount of time required for polymerization, of a 2-octyl cyanoacrylate composition is slightly longer when the cyanoacrylate composition is dispensed through a tip having a benzalkonium chloride initiator disposed thereon using methanol as compared to using acetone.

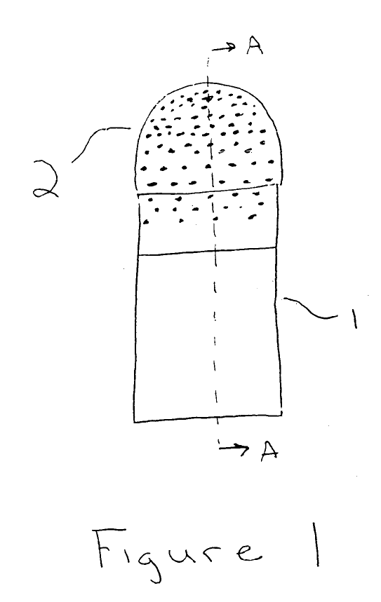

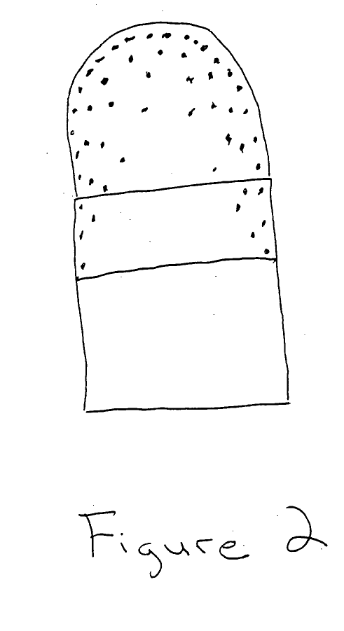

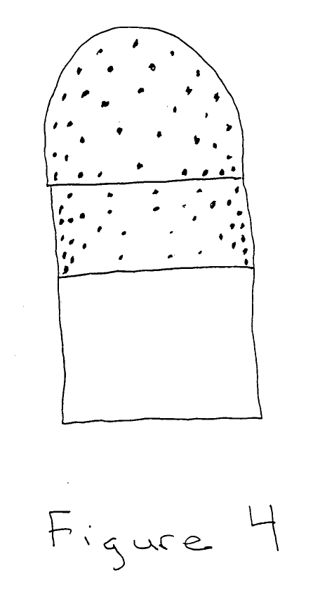

FIGS. 1-4 show effects of different solvents used to apply a polymerization initiator or a polymerization rate modifier to an applicator tip. The initiator or rate modifier is applied to the tip by pumping a liquid medium comprising the initiator or rate modifier through a syringe and onto the distal end of the tip. FIG. 1 shows an applicator tip 2 of applicator 1 treated with a solution of an initiator or rate modifier dissolved in 110 μL of methanol, and subsequently dried for about 30 minutes. FIG. 2 shows a cross-section of the applicator and tip in FIG. 1. The initiator or rate modifier is present on the tip in a concentration gradient. The initiator or rate modifier concentration is highest at the top, or distal, end of the applicator tip and decreases towards the center and bottom, or proximal, end of the applicator tip. In contrast, FIG. 3 shows an applicator tip treated with a solution of an initiator or rate modifier dissolved in 110 μL of acetone, and subsequently dried for about 30 minutes. FIG. 4 shows a cross-section of the applicator and tip in FIG. 3. The initiator or rate modifier is present on the tip in a concentration gradient that increases from the top (distal) end towards the bottom (proximal) end of the applicator tip.

The pattern of distribution of the material within the tip, when applied using a solvent comprising methanol, provides unexpectedly superior polymerization characteristics to monomer compositions dispensed through the tip as compared to compositions dispensed through tips prepared using other low boiling point solvents, such as acetone. The monomer compositions dispensed through tips prepared using solvents comprising methanol polymerize rapidly enough to make them convenient to apply; however, the polymerization that occurs does not result in the level of tissue damage often seen when other systems of cyanoacrylate delivery are used.

According to this aspect of the present invention, the solvent (also referred to herein as the liquid medium) for application of the material comprises methanol. Methanol can be used as the only solvent, or it can be present in mixtures of methanol with other low boiling point solvents, including low molecular weight ketones such as acetone. The mixture can be in any ratio. Preferably, the mixture is a ratio of methanol to other solvent of between 99:1 to 1:99. For example, the ratio of methanol to other solvent can be approximately 80:20 to 20:80, or 60:40 to 40:60. A ratio of at least 70:30, such as 80:20 or 90:10. or higher, is desirable in embodiments. Preferably, when the solvent comprises a component other than methanol, the other component is a low boiling point solvent having a vapor pressure of about 25-150 mm Hg at 20° C., such as about 30-125 mm Hg at 20° C., or about 40-100 mm Hg at 20° C., or mixtures thereof. In embodiments, low molecular weight solvents can be used. Included are low molecular weight ketones and alcohols. However, in other embodiments of the invention, a material may be applied to an applicator tip with other solvents, including water.

The material may be applied to the applicator tip by spraying, dipping, injecting, or brushing the applicator tip with a liquid medium containing the material. It is preferably applied to the tip by dipping or injecting. For example, it may be applied to the tip by pumping of the liquid medium, for example, through a syringe, onto the distal end of the tip.

In embodiments where the applicator tips are porous, the material may be applied to the applicator tip by using a vacuum or pressure process. In each process, a solution or suspension of the material is introduced into a vacuum or pressure chamber. The applicator tips, either individually or preferably in batches, are placed into the solution or suspension in the pressure vessel in a manner such that the applicator tips preferably do not float to the top of the solution or suspension. For example, the applicator tips can be placed in the solution or suspension in a wire basket or other suitable container, which would hold the applicator tips under the solution or suspension, or a wire mesh or other suitable retainer could be placed over the applicator tips to dunk or sink them into the solution or suspension. Once the applicator tips are in the solution or suspension, the vessel can be sealed and an appropriate vacuum or pressure applied.

Application of the vacuum or pressure results in air that is trapped in the applicator tips being degassed, or forced out of the applicator tips, and being replaced by the solution or suspension. This replacement of air by the solution or suspension thereby loads the material onto or into the applicator tips. The end of the degassing phase can be observed by the absence of newly formed air bubbles. After a desired treatment time, the vacuum or pressure in the vessel can be released, and the treated applicator tips can be removed.

In exemplary embodiments, preparing an applicator for dispensing polymerizable monomeric compositions includes applying a material to a suitable applicator tip, such as a porous polyethylene tip or a foam or fibrous swab, which is attached to an applicator body, such as a butyrate applicator tube or an applicator handle such as a plastic, wood, metal or other suitable material handle or holder.

When the applicator is intended to contain an amount of polymerizable monomeric composition, the applicator body or tube may comprises a conduit or reservoir for the polymerizable monomeric composition. In this embodiment, the applicator tip may be operably connected to the conduit or reservoir, such as by being fitted on an open end of the conduit, so that fluid flowing through the conduit also flows through the applicator tip. In other embodiments, however, the applicator body may be free of an adhesive reservoir and may be intended to function only as a handle by which to grip the applicator, without itself containing the polymerizable monomeric composition. In these embodiments, for example, the applicator body can be a solid or hollow tube, such as a pipe, stick, rod, dowel, or the like, either straight or contoured. Such embodiments can be, for example, intended to apply a polymerizable monomeric composition by dipping the applicator into the polymerizable monomeric composition or dripping the monomeric composition onto the tip, rather than forcing the polymerizable monomeric composition through the applicator tip from the applicator handle.

Use of solvents comprising methanol, for example, also provides adequate bonding of the butyrate applicator tube or applicator body to a polyethylene applicator tip. The solvent used to apply the material to the tip also helps bond the polyethylene applicator tip to the butyrate applicator tube or applicator body. When using acetone, damage to the tube, applicator body and/or tip can occur if too much acetone is used. Solvents comprising methanol, while still providing the bonding that is necessary to hold the tip to the tube or applicator, allow the use of a greater range of solvent amounts to apply the material to the tip.

An anisotropic distribution or a concentration gradient of material in the applicator tip can be obtained with the use of a methanol-containing solvent. The distribution of the material may be varied depending on the solvent or solvents used to apply it and on the wetting characteristics of the solvent and tip. In general, the wetting characteristics of the solvent should be such that the surface tension is close enough to that of the tip material to wet at least the surface of the tip.

The material applied to the applicator tip can be any material, but is preferably an initiator that initiates polymerization and/or cross-linking of the monomer; a polymerization rate modifier, which modifies the rate of polymerization of the monomer; a bioactive material, such as a medicament; and/or a flavorant. The material may be applied to a surface portion or to the entire surface of the applicator tip. Preferably, only a portion of the exterior of the applicator tip is treated with the material.

Particular initiators and rate modifiers for particular monomers may be readily selected by one of skill in the art without undue experimentation. Control of the molecular weight distribution of the applied adhesive can be enhanced by selection of the concentration and functionality of the initiator or rate modifier vis-a-vis the selected monomer. Suitable polymerization initiators and rate modifiers for cyanoacrylate compositions include, but are not limited to, detergent compositions; surfactants, including nonionic surfactants such as polysorbate 20 (e.g., Tween 20™; ICI Americas), polysorbate 80 (e.g., Tween 80™; ICI Americas), and poloxamers; cationic surfactants such as tetrabutylammonium bromide; anionic surfactants, including quaternary ammonium halides such as benzalkonium chloride or its pure components, and benzethonium chloride; stannous octoate (tin (II) 2-ethylhexanoate), and sodium tetradecyl sulfate; and amphoteric or zwitterionic surfactants such as dodecyldimethyl(3-sulfopropyl) ammonium hydroxide, inner salt; amines, imines, and amides, such as imidazole, tryptamine, urea, arginine and povidine; phosphines, phosphites and phosphonium salts, such as triphenylphosphine and triethyl phosphite; alcohols such as ethylene glycol; methyl gallate; ascorbic acid; tannins and tannic acid; inorganic bases and salts, such as sodium bisulfite, magnesium hydroxide, calcium sulfate and sodium silicate; sulfur compounds such as thiourea and polysulfides; polymeric cyclic ethers such as monensin, nonactin, crown ethers, calixarenes and polymeric epoxides; cyclic and acyclic carbonates, such as diethyl carbonate; phase transfer catalysts such as Aliquat™ 336 (General Mills, Inc., Minneapolis, Minn.); organometallics; manganese acetylacetonate; radical initiators and radicals, such as di-t-butyl peroxide and azobisisobutyronitrile; and bioactive compounds or agents.

In preferred embodiments, the initiator may be a bioactive material, including quaternary ammonium halides such as alkylbenzyldimethylammonium chloride (benzalkonium chloride; BAC) its pure components, or mixtures thereof, especially those with an alkyl containing 6-18 carbon atoms; benzethonium chloride; and salts of sulfadiazine. Cobalt napthenate can be used as an accelerator for peroxide.

In preferred embodiments, the initiator may be a bioactive material that possesses antiviral, antimicrobial, antifungal and/or wound healing properties. An example of such a material that possesses polymerization initiation and antiviral, antimicrobial, and/or antifuiigal properties is Gentian Violet, also known as crystal violet or methylrosaniline chloride. Examples of materials that possess polymerization initiation and wound healing properties also include various zinc complexes and zinc salts, antioxidants such as vitamin E and other vitamins and the like, and copper compounds such as copper chloride, copper sulfate and copper peptides, as described in “Copper: An Essential Element for Life,” ProCyte Corporation, available at http://www.humatech.com/technology.html (Oct. 28, 1999), the entire disclosure of which is incorporated herein by reference. Such materials are particularly preferred because they can serve not only as the polymerization initiator or rate modifier for the cyanoacrylate monomer, they can also provide additional benefits to the wound site, such as antiviral effects, antimicrobial effects and/or antifungal effects or help to promote wound healing.

When present, the zinc compound can be present in various forms, such as zinc salts. For example, suitable zinc compounds include, but are not limited to, zinc salts of cyanoacrylic acid, zinc salts of cyanoacetic acid, zinc salts of dicyanoglutaric acid, zinc salts of rosin, zinc oxide, zinc salts of polycyanoacrylic acid, zinc salts of polyacrylic acid, zinc bacitracin, zinc salicylate, zinc stearate, zinc citrate, zinc lactate, mixtures thereof, and the like. Preferably, the zinc compounds are of Zn²⁺. Incorporation of such zinc compounds into the applied cyanoacrylate composition, either prior to or concurrent with application and/or initiation, is particularly effective in promoting wound healing of leg ulcers, thermal burns, and the like.

In embodiments where an antiviral, antimicrobial and/or antifungal material is used, crystal violet is particularly preferred. Crystal violet has many benefits, particularly when used in conjunction with the adhesive monomer compositions of the present invention. One benefit of crystal violet is that in addition to providing the antiviral, antimicrobial and/or antifungal effects, it also provides a visible color at the site of application, which can help ensure that a sufficient or desired amount of adhesive has been applied. However, whereas crystal violet is known to leave “tattoo” scars on tissue when it is applied, such tattoo scarring does not result when it is used in combination with the adhesive monomer compositions of the present invention. Rather, the crystal violet provides its coloring and other effects, without leaving a long-term or permanent mark. Furthermore, the crystal violet can be incorporated in the applicator tip in various amounts, to provide different results. For example, it can be incorporated in small amounts, such as that amount necessary to provide the desired amount of polymerization initiation, without leaving significant amounts in the polymerized compositions, and thus without providing significant or effective antiviral, antimicrobial and/or antifungal effect. In this case, the crystal violet provides the composition with a visible color prior to and during the polymerization, but the color fades as the crystal violet is consumed in polymerization. On contrast, a larger amount of crystal violet can be incorporated into the applicator tip, such that an effective amount of the material remains even after polymerization to provide the desired antiviral, antimicrobial and/or antifungal effects.

Furthermore, the mode of application of the crystal violet to the applicator tip can be varied to obtain desired composition gradients of the material in the applicator tip. For example, if it is desired to maintain the crystal violet only one or near the surface of the applicator tip, then it can be applied, for example, with acetone as described above. Alternatively, if it is desired to incorporate the crystal violet more evenly throughout the applicator tip, then it can be applied, for example, with methanol as also described above. In embodiments, the crystal violet, and/or other agents being added to the applicator tip, can be applied using water as a solvent.

The polymerizable and/or cross-linkable material may also contain an initiator and/or a rate modifier which is inactive until activated by a catalyst or accelerator (included within the scope of the term “initiator” as used herein) in the applicator tip. Initiators activated by stimulation such as heat and/or light (e.g., ultraviolet or visible light) are also suitable if the tip and/or applicator is appropriately subjected to such stimulation.

The initiator or rate modifier may be dissolved or otherwise dispersed in the solvent and applied to the applicator tip in any effective amount. An effective amount is that amount of initiator or rate modifier that effects polymerization to a gel point on dry tissue in less than 300 seconds, preferably within 150 seconds, and more preferably within 130 seconds, at ambient temperature, such as approximately 21-25° C. The coated applicator is then allowed to dry, thereby evaporating the solvent. In embodiments, the applicator is allowed to dry for about 5 to 35 minutes. In embodiments, the amount of initiator or modifier dissolved or dispersed in the solvent may be about, or less than, 25 wt. %, preferably less than 10 wt. % and more preferably less than 1 wt. %. The amount of initiator or rate modifier dissolved or dispersed in the solvent may be any effective amount. In the case where a quaternary ammonium halide is the initiator or rate modifier, the effective amount is preferably between 100 and 250 ppm or more in 110 μl. In the case of salts of sulfadiazine, the effective amount is preferably approximately 50 ppm or more in 110 μl. The effective amount for each initiator and adhesive monomer combination can easily be determined by one of ordinary skill in the art.

To determine the polymerization time, an appropriate volume of a solution of the initiator prepared in a volatile solvent is placed in a differential scanning calorimetric pan. The volatile solvent is allowed to dry under ambient conditions. Alternatively, the appropriate quantity of the initiator is dispensed directly onto the differential scanning calorimetric pan. In either of the abovementioned cases, 25 μl of the chosen monomer solution is pipetted into the pan. The time taken for the monomer composition to polymerize to the point of a gel is the polymerization time.

In embodiments, the initiator and/or the rate modifier can be, but does not have to be, bioactive. In embodiments where the initiator and/or the rate modifier is bioactive, the method of the invention can be used to close, cover, or protect tissue and wounds while simultaneously providing a bioactive material to the tissue or wound.

In embodiments where the initiator is also a bioactive material, the bioactive material is applied onto the tip in an amount that is effective to initiate polymerization and to be effective for the biological activity intended (e.g., in a sufficient amount to be antiseptic). The bioactive material is selected in conjunction with the polymerizable monomer to be dispensed such that the bioactive material functions as an initiator or rate modifier for the monomer. During dispensing of the monomer composition, the bioactive material is mixed with the monomer composition. In embodiments, the bioactive material can be released to the tissue to be treated at a constant, or near constant, rate over a period of time while the polymerized composition is in contact with the wound site.

As mentioned above, the bioactive material can, but need not, be a polymerization initiator or rate modifier. Where the bioactive material is not an initiator or a rate modifier, an initiator or rate modifier can also be applied to the tip along with the bioactive material. In such a situation, it is not critical that the bioactive material be distributed anisotropically or in a gradient along the applicator tip. In embodiments where the bioactive material is not an initiator, it can be applied to the applicator tip in a solution comprising a low boiling point solvent, especially one comprising methanol. However, any suitable solvent (including water and other aqueous solvents) can be used to apply the bioactive material. In embodiments where the applicator tip contains a bioactive material, the bioactive material is solubilized, dissolved, or otherwise dispersed in the adhesive composition as the composition enters and leaves the tip. Thus, the bioactive material similarly mixes with the adhesive composition prior to, and during, application of the adhesive. Coapplication of the bioactive material and adhesive composition allows this mixing to occur. Further mixing can occur once the adhesive composition bioactive material has been dispensed at the wound site. Such coapplication (e.g., coelution) of the bioactive material and the adhesive composition provides an advantage not disclosed in the prior art.

Suitable bioactive materials include, but are not limited to, medicaments such as antibiotics, antimicrobials, antiseptics, bacteriocins, bacteriostats, disinfectants, steroids, anesthetics, antifungal agents, anti-inflammatory agents, antibacterial agents, antiviral agents, antitumor agents, growth promoting substances, antioxidants, or mixtures thereof. Such compounds include, but are not limited to, acetic acid, aluminum acetate, bacitracin, bacitracin zinc, benzalkonium chloride, benzethonium chloride, betadine, calcium chloroplatinate, certrimide, cloramine T, chlorhexidine phosphanilate, chlorhexidine, chlorhexidine sulfate, chloropenidine, chloroplatinatic acid, ciprofloxacin, clindamycin, clioquinol, cysostaphin, gentamicin sulfate, hydrogen peroxide, iodinated polyvinylidone, iodine, iodophor, minocycline, mupirocin, neomycin, neomycin sulfate, nitrofurazone, non-onynol 9, potassium permanganate, penicillin, polymycin, polymycin B, polymyxin, polymyxin B sulfate, polyvinylpyrrolidone iodine, povidone iodine, 8-hydroxyquinoline, quinolone thioureas, rifampin, rifamycin, copper chloride, copper sulfate, copper peptides, silver acetate, silver benzoate, silver carbonate, silver chloride, silver citrate, silver iodide, silver nitrate, silver oxide, silver sulfate, sodium chloroplatinate, sodium hypochlorite, sphingolipids, tetracycline, zinc oxide, salts of sulfadiazine (such as silver, sodium, and zinc), antioxidants such as vitamins such as vitamin E, other agents mentioned above, and mixtures thereof. Preferable bioactive materials are USP approved, more preferably USP monographed.

As an alternative to using an additional polymerization initiator or rate modifier, it is possible to formulate the applicator such that the adhesive can be initiated by the tip structural material when it is applied to the desired surface. For examples, the applicator tip could be treated with a basic agent, after or preferably before or during its attachment to an applicator body. Treatment with such an agent, such as a caustic agent or alkyl hydroxide, can cause reticulation of the applicator tip material, which in turn results in an applicator tip that is self-initiating when the polymerizable material comes into contact with the applicator tip. In this embodiment, additional polymerization initiators or rate modifiers could be omitted, because a desired initiation and polymerization rate could be selected by proper treatment of the applicator tip material.

In this embodiment, the applicator tip material can be treated with any suitable agent, so long as the objectives of the invention are maintained. Suitable agents include, but are not limited to, caustic soda (NaOH), potassium hydroxide, other hydroxides of light metals, ammonium hydroxide, alkyl hydroxides, caustic alcohol (C₂H₅ONa), silver nitrate, other strongly alkaline materials, mixtures thereof, and the like.

In addition to the above materials, or in place thereof, the applicator tip can also include various other materials that may or may not act as a polymerization initiator or rate modifier. For example, the applicator tip can include a flavorant, such that it imparts a flavor to the adhesive material when the adhesive material is applied to a surface. Incorporation of a flavorant is particularly preferred, for example, when the cyanoacrylate adhesive material is to be applied to oral surfaces, such as to treat stomatitis or cold sores.

When a flavorant is to be included, any of the various available and suitable flavorants can be used. Suitable flavorants can be selected, for example, from among fruit oil, vegetable oil, esters, heterocyclic compounds, fruit extract and vegetable extract. In particular, the flavoring additive may be selected from among any of the various known flavoring additives, including, but not limited to, 5-fold orange oil (Florida Chemical Co.), anethole (Aldrich), banana distillate (Florida Chemical Co.), benzaldehyde (Aldrich), clove oil (Humco), cold pressed valencia orange oil (Florida Chemical Co.), cold pressed grapefruit oil (Florida Chemical Co.), cold pressed lemon oil (Florida Chemical Co.), cold pressed lime oil (Florida Chemical Co.), cucumber distillate (Florida Chemical Co.), honey distillate (Florida Chemical Co.), menthol (Aldrich), alkyl salicylates such as methyl salicylate (Lorann Oils or Aldrich), monosodium glutamate, spearmint, wintergreen, cinnamon, citrus, cherry, apple, peppermint, peppermint oil (Humco), peppermint spirit, vanillin (Aldrich), thymol (Aldrich), and ethyl vanillin, mixtures thereof, and the like. The flavorant can also be a sweetener, such as a suitable sugar or sugar substitute. Examples of such sweeteners include, but are not limited to, saccharin, sorbitol, mannitol, aspartame, sucrose, glucose, fructose, and the like. In preferred embodiments, the flavoring additive is a flavoring agent as defined in 21 C.F.R. §172.510, dated Jun. 12, 1989, and §172.515, dated Apr. 1, 1996, the entireties of which are incorporated herein by reference. Flavoring additives are also disclosed in U.S. patent application Ser. No. 09/343,914, filed Jun. 30, 1999, the entire disclosure of which is incorporated herein by reference.

The flavoring additive is selected such that it is preferably compatible with the monomer (i.e., does not adversely affect polymerization, bond strength, cure properties, or shelf-life). Preferably, the flavoring additive is soluble in the monomer composition at room temperature (i.e., 20-25° C.) so that it may be readily solubilized in the monomer composition while the monomer composition is in contact with or passing through the applicator tip. Furthermore, the flavoring additive is selected such that it is preferably compatible with the applicator tip and any other components that are to be incorporated into or on the applicator tip.

The flavoring additive is used in an amount to provide the desired flavor level to the final polymerized adhesive. For example, the flavorant can be provided in an amount of, for example, from about 0.001-25.0% by weight of the adhesive composition to be applied. In preferred embodiments, the flavoring additive is incorporated in an amount of from about 0.2-10.0%, more preferably 0.5-5.0%, of the adhesive composition. Of course, additive amounts outside of these ranges can be readily used depending upon, for example, the desired result to be achieved and the relative flavoring strength of the particular additive. The amount of flavoring additive to be used can be determined by one of ordinary skill in the art based on the present disclosure using known techniques without undue experimentation.

Furthermore, in embodiments, the flavorant can be applied in combination with a delivery substrate to facilitate incorporation of the flavorant into or on the applicator tip. Where used, suitable delivery substrates include, but are not limited to, waxes, such as carnauba, petroleum and carbowax; gels, such as gelatin, hydroxypropyl methylcellulose, carboxymethylcellulose, and hydroxy-gels; polyethylene glycol; polysorbate; agar; povidone; sodium stearate; starch; powdered sugar; high fructose corn syrup; fructose; glycerin; hydrogenated glucose syrup; sorbitol; mannitol; sucrose; cellulose acetate phthalate; dextrose; polyvinyl alcohol; mixtures thereof; and the like.

Still further, it may be desirable to incorporate a preservative into the applicator tip in addition to the flavorant, to help preserve and maintain the flavoring effect of the flavorant. The need for such a preservative can depend, for example, upon the concentration and nature of flavorant, or lack thereof, in the applicator tip. Suitable preservatives generally include the known food preservatives, such as sodium benzoate, salt, citric acid, benzoic acid, sodium nitrite, sodium phosphate, and the like.

Preferably, the flavorant, delivery substrate and/or preservative do not adversely affect the applicator tip. For example, it is preferred that these materials do not adversely affect the aging and/or shelf-life of the applicator tip.

The present invention is also directed to a method of applying the adhesive composition utilizing an applicator comprising a tip having a polymerization initiator, a polymerization rate modifier, a bioactive material and/or a flavorant thereon or therein. According to the invention, any appropriate design for the applicator can be used. Such applicator designs include, but are not limited to, swab applicators, syringes, adhesive guns, pipettes, eyedroppers, vials, and the like with various dispensing nozzles or tips. Suitable applicators may incorporate or be packaged, such as in saleable kits, with one or more containers containing the adhesive composition and/or other components.

For example, the applicator tip may be permanently fixed to or detachable from an applicator container holding the polymerizable and/or cross-linkable material. Such an applicator tip could be attached to the applicator container prior to use and detached from the applicator container subsequent to use in order to prevent premature polymerization or cross-linking of the unapplied material in the applicator container. At this point the applicator tip may be discarded and a new applicator tip may be attached to the applicator container for subsequent use, or the applicator tip may be cleaned and reused.

As a further example, the applicator tip can be a swab attached to a suitable applicator body, such as a plastic, wood, metal or other suitable material handle or holder. Such an applicator can be used, for example, to apply adhesive material from a separate container. The adhesive material can be applied by dipping the swab into the adhesive material, or by otherwise transferring the adhesive material to the swab, and then applying the adhesive material to the desired surface.

In this embodiment, the applicator including the swab tip can be provided separately, or as part of a saleable kit that includes both the applicator and a quantity of adhesive material, which may be either operably connected to the applicator tip or swab, or located in a separate container. Various designs of such kits are disclosed, for example, in U.S. patent application Ser. No. 09/385,030, filed Aug. 30, 1999 the entire disclosure of which is incorporated herein by reference. In such embodiments. the applicator tip can include any or all of the various materials described above. Preferably the applicator tip in such swab embodiments includes a polymerization initiator or rate modifier that may also be a bioactive material and/or a flavorant.

Additionally, the applicator tip according to the present invention may comprise multiple parts, with at least one part having the initiator, rate modifier, bioactive material and/or flavorant. For example, the component containing the initiator, rate modifier, bioactive material and/or flavorant may be fabricated separately from the other component(s) of the applicator tip and assembled prior to attachment to the applicator body or container.

The applicator tip may also be in the form of a nozzle for atomizing liquid polymerizable and/or cross-linkable materials. Conical, flat spray or condensed stream nozzles are suitable.

The applicator tip and the applicator container may be an integral or even monolithic unit. The unit may be preformed as a single piece and charged with polymerizable and/or cross-linkable material. After application of material from the applicator container, the unit may be discarded. Additionally, such an integral or monolithic applicator tip/applicator container unit may be fashioned to provide the capability of recharging the unit with new material as a multiple use device.

The applicator tip may be composed of any of a variety of materials including polymerized materials such as plastics, foams, rubber, thermosets, films, fibers, or membranes. Where foams are used in the applicator tip, the foam can be either an open-celled form, a closed-cell foam, or a mixture thereof. Any suitable foam material can be used and include, for example, thermoplastic polyurethane foam. In swab tip embodiments, the foam is preferably a soft, absorbent thermoplastic polyurethane foam.

In embodiments, the applicator tip may be made from polyurethane, polyesters, polyolefins such as polyethylene, or polyamides. In embodiments, the applicator may be made from polyethylene, such as that sold by Porex Technologies Corp. (Fairburn, Ga.) under the name LabPor®. In embodiments, the applicator tip can also be made from fibers, either natural or synthetic, such as cotton, rayons, nylons, and mixtures thereof. Additionally, the applicator tip may be composed of materials such as metal, glass, paper, ceramics, and the like. The applicator tip material may be porous, absorbent, or adsorbent in nature to enhance and facilitate loading of a material on or in the applicator tip. For example, the applicator tip may be composed of a material having random pores, capillaries, a honeycomb material, a material having a woven pattern, etc. The degree of porosity will depend on the materials being used, and can be determined easily by one of ordinary skill in the art. Porosity is the open volume within the pores of an applicator tip divided by the total volume of the applicator tip.

In embodiments, the applicator tip may be porous and have an average pore size of about 1 μm to about 500 μm. Generally, according to the present invention, an applicator tip having an average pore size of about 1-100 μm such as 10-30 is used with a polymerizable material having a viscosity of about 1-30 cPs, preferably about 2-18 cPs, and more preferably 5-7 cPs at 25° C. An applicator tip having an average pore size of from about 1 μm to about 100 μm is preferably used with a polymerizable material having a viscosity of about 10-30 cPs. When the polymerizable and/or cross-linkable material has a viscosity higher than 7 cPs, the average pore size of the applicator tip is generally increased. For example, an applicator tip having an average pore size of about 100-200 μm such as 140 μm is preferably used with a polymerizable material having a viscosity of about 30-500 cPs, preferably about 35-350 cPs, and more preferably about 200-300 cPs at 25° C. In embodiments, an applicator tip has a porosity of less than or equal to 80 percent.

In embodiments, when using a porous applicator, the amount of initiator or rate modifier necessary to initiate and/or to modify the rate of polymerization and/or cross-linking increases as the pore size of the applicator tip increases.

The applicator tip can have a variety of suitable shapes and sizes. Generally, the dimensional characteristics are limited only by the intended use of the applicator, and practicality considerations. Suitable shapes include, but are not limited to, conical, cylindrical, chisel or polygonal shapes. The length and size of the tip can be varied depending on various application parameters. The tip may be detachable from the applicator body, or may be an integral part of the applicator.

The applicator tip according to the present invention, where it connects to the applicator tube, may have an elongated tubular portion, out of which the mixed polymerizing and/or cross-linking material is expelled. A portion of the applicator tip which is immediately downstream of the applicator tube is advantageously porous in order to avoid a sharp pressure drop and ensure a constant mixed ratio profile. The structure can preferably trap fragments of any barriers or materials used to separate one or more components within the applicator container so that they will not clog the device or contact the patient.

When using a porous applicator tip to apply the adhesive composition, the composition preferably is not expressed directly through the applicator tip in a continuous motion. According to embodiments of the present invention, the adhesive composition is (1) expressed to the end or part way to the end of the applicator tip, (2) the pressure is released to draw the composition back into the applicator, and (3) the composition is then subsequently expressed through the applicator tip in a continuous motion. This is called a suck-back method of applying the adhesive composition of the present invention. When used with a tip that bears an initiator, this method lets the adhesive composition polymerize more slowly than if it had been expressed directly through the tip.

The initiator, rate modifier, bioactive material and/or flavorant may be in the form of a solid, such as a powder or a solid film, or in the form of a liquid, such as a viscous or paste-like material. The tip may also include a variety of additives, such as surfactants or emulsifiers. Preferably, the initiator, rate modifier, bioactive material and/or flavorant is soluble or otherwise dispersible in the polymerizable and/or cross-linkable material, and/or comprises or is accompanied by at least one surfactant which helps it co-elute with the polymerizable and/or cross-linkable material. In embodiments, the surfactant may help solubilize it in the polymerizable and/or cross-linkable material. The initiator, rate modifier, bioactive agent and/or flavorant thus mixes with the adhesive composition as the mixture passes through the tip.

The present invention provides a method of wound treatment, including wound closure. The methods of this invention can be used as replacements for, or in addition to, sutures or staples to join together two surfaces by applying the present compositions to opposing wound surfaces that are then held together while polymerization proceeds. The methods of this invention can also be used to coat, protect, or otherwise cover surface, superficial, or otherwise topical wounds including, but not limited to, minor cuts, scrapes, irritations, compromised skin, superficial lacerations, abrasions, burns, sores, and stomatitis. The methods of the invention can also be used on tissues that do not show any signs of tissue damage. For example, the methods can be used to deliver medicaments to a patient through healthy tissue. They can also be used, for example, to locally deliver medicaments to tissues such as tumors or organs.

In embodiments, the present invention provides a replacement for sutures or staples and includes a method of joining together in vivo two surfaces, comprising: (a) holding together tissue surfaces of a wound or incision to form an abutted tissue surface; (b) applying to the abutted tissue surface a composition of the present invention; and (c) maintaining the surfaces in contact until the composition polymerizes. A subsequent coating may be applied immediately after application of a previous coating or after a previous coating has been completely polymerized. Preferably, the monomer composition applied to the abutted tissue surface is allowed to at least partially polymerize prior to subsequent coatings or applications of additional monomer composition. A coating of an adhesive composition of the present invention having a monomer different from the monomer of the first or previous coating may be applied as the second or subsequent coating.

Repairing injured tissues (for example, to control bleeding) comprises, in general, sponging to remove superficial body fluids, holding injured tissue surfaces together in an abutting relationship and subsequent application to the exposed abutted tissue of the present adhesive composition. The composition polymerizes to a thin film of polymer while in contact with the abutted tissue surface. Tissues which are not bleeding or otherwise covered by body fluids need not be sponged first. More than one coating or application of monomer composition may be applied to the abutted tissue surface. Desired bonding of tissues or hemostasis proceeds well in the presence of blood and other body fluids. The bonds formed are of adequate flexibility and strength to withstand normal movement of tissue. In addition, bond strength is maintained as natural wound healing proceeds.

In embodiments, the present invention is directed to a method of treating a superficial or topical wound, such as a skin wound or a wound on a mucous membrane which comprises (a) applying a composition of the present invention to the superficial wound; (b) allowing the composition to polymerize; and (c) optionally, applying the composition at least once more to the coated superficial wound.

The presence of a plasticizing agent and/or an acidic stabilizing agent can cause such a coating to have sufficient bond strength and flexibility, even with significant film or coating thicknesses. Suitable film thickness for wound closure in the cases where the adhesive is replacing sutures range from 0.1 mm to 2.0 mm or 3.0 mm or higher, preferably from 0.2 mm to 1.5 mm, and more preferably from 0.4 mm to 0.8 mm. Suitable film thickness for other applications range from 1 μm to 1000 μm.

In embodiments, the biocompatible film formed for replacement of sutures may have an in vivo film strength of at least 70 mm Hg of vacuum pressure required to induce wound failure, generally from 70 mm Hg to 400 mm Hg of vacuum pressure required to induce wound failure, preferably from 90 mm Hg to 400 mm Hg of vacuum pressure required to induce wound failure, and more preferably from 100 mm Hg to 400 mm Hg of pressure required to induce wound failure. In embodiments, the biocompatable film formed for other applications may have an in vivo film strength of 5-400 mm Hg of vacuum pressure required to induce wound failure, more preferably 50-400 mm Hg.

Although the invention has been described above as incorporating the initiator or rate modifier material, and/or other materials, into a pre-formed applicator tip, the present invention is not limited to such embodiments. In particular, according to embodiments of the present invention, the material or materials can be introduced into or onto the applicator tip during the process of manufacturing the applicator tip. Thus, in such embodiments, rather than applying the initiator or other material using a solvent that subsequently evaporates, the initiator and/or other material is incorporated directly into or onto the applicator tip during manufacture of the tip.

The initiator and/or other material can be incorporated into the applicator at any suitable stage during the manufacturing process. For example, where the applicator tip is made by molding pellets of a polymeric substance, the material can be incorporated into the applicator tip prior to, concurrent with or subsequent to molding of the applicator tip. For example, the material can be mixed with the pellets used to form the applicator tip, such that the mixture is molded to form the applicator tip. Alternatively, where the material is a liquid or can be dissolved into a suitable carrier liquid, the material can be absorbed into or adsorbed onto the pellets prior to molding, or can be applied as a release agent to the mold. An advantage of using foams for the applicator tip is that the materials described herein can be incorporated into the foam during or after the foam formation. The materials can be incorporated into the foam, for example, by introducing them into the foam during the blowing process, by adding them as a release agent to remove the foam from a mold, and the like. These processes provide alternative means to incorporate the initiator or other material into or onto the applicator tip in a controlled manner, without need for a subsequent step of applying the material to the pre-formed applicator tip.

The present invention involves a polymerizable adhesive composition, such as a monomer composition comprising:
A) at least one polymerizable monomer that forms a medically acceptable adhesive polymer;
B) optional plasticizing agents;
C) optional stabilizing agents; and
D) optional thickening agents.

In embodiments, the composition preferably comprises a monomeric (or prepolymeric) adhesive. In embodiments, the monomer is a 1,1-disubstituted ethylene monomer, for example, an α-cyanoacrylate. In embodiments, the monomer composition comprises a bioactive material. Preferred compositions of the present invention and polymers formed therefrom are useful as tissue adhesives, sealants for preventing bleeding or for covering open wounds, and in other biomedical applications. They find uses in, for example, apposing surgically incised or traumatically lacerated tissues; retarding blood flow from wounds; drug delivery; dressing burns; dressing skin or other superficial or surface wounds (such as abrasions, chaffed or raw skin, and/or stomatitis); protecting tissues prone to damage (e.g., as artificial calluses); and aiding repair and regrowth of living tissue.

U.S. Pat. No. 5,328,687 to Leung et al; U.S. Pat. No. 3,527,841 to Wicker et al.; U.S. Pat. No. 3,722,599 to Robertson et al.; U.S. Pat. No. 3,995,641 to Kronenthal et al.; and U.S. Pat. No. 3,940,362 to Overhults; and U.S. patent applications Ser. Nos. 08/266,647 and 09/099,457, disclose materials that are useful as surgical adhesives. All of the foregoing references are hereby incorporated in their entirety by reference.

Monomers that may be used in this invention are readily polymerizable, e.g. anionically polymerizable or free radical polymerizable, or polymerizable by zwitterions or ion pairs to form polymers. Such monomers include those that form polymers that may, but do not need to, biodegrade. Such monomers are disclosed in, for example, U.S. Pat. No. 5,328,687, which is hereby incorporated in its entirety by reference herein.

Useful 1,1-disubstituted ethylene monomers include, but are not limited to, monomers of the formula:
HRC═CXY (I)
wherein X and Y are each strong electron withdrawing groups, and R is H, —CH═CH₂ or, provided that X and Y are both cyano groups, a C₁-C₄ alkyl group.

Examples of monomers within the scope of formula (I) include α-cyanoacrylates, vinylidene cyanides, C₁-C₄ alkyl homologues of vinylidene cyanides, dialkyl methylene malonates, acylacrylonitriles, vinyl sulfinates and vinyl sulfonates of the formula CH₂═CX′Y′ wherein X′ is —SO₂R′ or —SO₃R′ and Y′ is —CN, —COOR′, —COCH₃, —SO₂R′ or —SO₃R′, and R′ is H or hydrocarbyl.

Preferred monomers of formula (I) for use in this invention are α-cyanoacrylates. These monomers are known in the art and have the formula 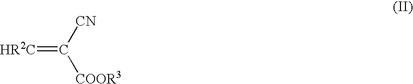
wherein R² is hydrogen and R³ is a hydrocarbyl or substituted hydrocarbyl group; a group having the formula —R⁴—O—R⁵—O—R⁶ or the formula —R⁵—O—R⁶, wherein R⁴ is a 1,2-alkylene group having 2-4 carbon atoms, R⁵ is an alkylene group having 2-4 carbon atoms, and R⁶ is an alkyl group having 1-6 carbon atoms; or a group having the formula 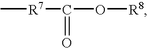
wherein R⁷ is 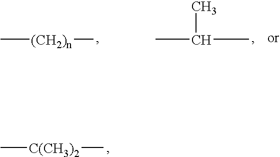
wherein n is 1-10, preferably 1-5 carbon atoms, and R⁸ is an organic radical.

Examples of suitable hydrocarbyl and substituted hydrocarbyl groups include straight chain or branched chain alkyl groups having 1-16 carbon atoms; straight chain or branched chain C₁-C₁₆ alkyl groups substituted with an acyloxy group, a haloalkyl group, an alkoxy group, a halogen atom, a cyano group, or a haloalkyl group; straight chain or branched chain alkenyl groups having 2 to 16 carbon atoms; straight chain or branched chain alkynyl groups having 2 to 12 carbon atoms; cycloalkyl groups; aralkyl groups; alkylaryl groups; and aryl groups.

The organic moiety R⁸ may be substituted or unsubstituted and may be straight chain, branched or cyclic, saturated, unsaturated or aromatic. Examples of such organic moieties include C₁-C₈alkyl moieties, C₂-C₈ alkenyl moieties, C₂-C₈ alkynyl moieties, C₃-C₁₂ cycloaliphatic moieties, aryl moieties such as phenyl and substituted phenyl and aralkyl moieties such as benzyl, methylbenzyl and phenylethyl. Other organic moieties include substituted hydrocarbon moieties, such as halo (e.g., chloro-, fluoro- and bromo-substituted hydrocarbons) and oxy- (e.g., alkoxy substituted hydrocarbons) substituted hydrocarbon moieties. Preferred organic radicals are alkyl, alkenyl and alkynyl moieties having from 1 to about 8 carbon atoms, and halo-substituted derivatives thereof. Particularly preferred are alkyl moieties of 4 to 6 carbon atoms.

In the cyanoacrylate monomer of formula (II), R³ is preferably an alkyl group having 1-10 carbon atoms or a group having the formula -AOR⁹, wherein A is a divalent straight or branched chain alkylene or oxyalkylene moiety having 2-8 carbon atoms, and R⁹ is a straight or branched alkyl moiety having 1-8 carbon atoms.

Examples of groups represented by the formula -AOR⁹ include 1 -methoxy-2-propyl, 2-butoxy ethyl, isopropoxy ethyl, 2-methoxy ethyl, and 2-ethoxy ethyl.

Preferred α-cyanoacrylate monomers used in this invention include 2-octyl cyanoacrylate, dodecyl cyanoacrylate, 2-ethylhexyl cyanoacrylate, butyl cyanoacrylate, methyl cyanoacrylate, 3-methoxybutyl cyanoacrylate, 2-butoxyethyl cyanoacrylate, 2-isopropoxyethyl cyanoacrylate, or 1-methoxy-2-propyl cyanoacrylate.

The α-cyanoacrylates of formula (II) can be prepared according to methods known in the art. U.S. Pat. Nos. 2,721,858 and 3,254,111, each of which is hereby incorporated by reference herein, disclose methods for preparing α-cyanoacrylates. For example, the α-cyanoacrylates can be prepared by reacting an alkyl cyanoacetate with formaldehyde in a non-aqueous organic solvent and in the presence of a basic catalyst, followed by pyrolysis of the anhydrous intermediate polymer in the presence of a polymerization inhibitor. The α-cyanoacrylate monomers prepared with low moisture content and essentially free of impurities are preferred for biomedical use.

The α-cyanoacrylates of formula (II) wherein R³ is a group having the formula R⁴—O—R⁵—O—R⁶ or the formula —R⁵—O—R⁶ can be prepared according to the method disclosed in U.S. Pat. No. 4,364,876 to Kimura et al., which is hereby incorporated by reference. In the Kimura et al. method, the α-cyanoacrylates are prepared by producing a cyanoacetate by esterifying cyanoacetic acid with an alcohol or by transesterifying an alkyl cyanoacetate and an alcohol; condensing the cyanoacetate and formaldehyde or para-formaldehyde in the presence of a catalyst at a molar ratio of 0.5-1.5:1, preferably 0.8-1.2:1, to obtain a condensate; depolymerizing the condensation reaction mixture either directly or after removal of the condensation catalyst to yield crude cyanoacrylate; and distilling the crude cyanoacrylate to form a high purity cyanoacrylate.

The α-cyanoacrylates of formula (II) wherein R³is a group having the formula 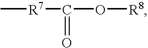
can be prepared according to the procedure described in U.S. Pat. No. 3,995,641 to Kronenthal et al., which is hereby incorporated by reference. In the Kronenthal et al. method, such α-cyanoacrylate monomers are prepared by reacting an alkyl ester of an α-cyanoacrylic acid with a cyclic 1,3-diene to form a Diels-Alder adduct which is then subjected to alkaline hydrolysis followed by acidification to form the corresponding α-cyanoacrylic acid adduct. The α-cyanoacrylic acid adduct is preferably esterified by an alkyl bromoacetate to yield the corresponding carbalkoxymethyl α-cyanoacrylate adduct. Alternatively, the α-cyanoacrylic acid adduct may be converted to the α-cyanoacrylyl halide adduct by reaction with thionyl chloride. The α-cyanoacrylyl halide adduct is then reacted with an alkyl hydroxyacetate or a methyl substituted alkyl hydroxyacetate to yield the corresponding carbalkoxymethyl α-cyanoacrylate adduct or carbalkoxy alkyl α-cyanoacrylate adduct, respectively. The cyclic 1,3-diene blocking group is finally removed and the carbalkoxy methyl α-cyanoacrylate adduct or the carbalkoxy alkyl α-cyanoacrylate adduct is converted into the corresponding carbalkoxy alkyl α-cyanoacrylate by heating the adduct in the presence of a slight deficit of maleic anhydride.

Examples of monomers of formula (II) include cyanopentadienoates and β-cyanoacrylates of the formula: 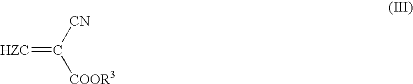
wherein Z is —CH═CH₂ and R³ is as defined above. The monomers of formula (III) wherein R³ is an alkyl group of 1-10 carbon atoms, i.e., the 2-cyanopenta-2,4-dienoic acid esters, can be prepared by reacting an appropriate 2-cyanoacetate with acrolein in the presence of a catalyst such as zinc chloride. This method of preparing 2-cyanopenta-2,4-dienoic acid esters is disclosed, for example, in U.S. Pat. No. 3,554,990, which is hereby incorporated by reference herein.

Preferred monomers are alkyl α-cyanoacrylates and more preferably octyl α-cyanoacrylates, especially 2-octyl α-cyanoacrylate. Monomers utilized in the present application should be very pure and contain few impurities (e.g., surgical grade).

When present, component B) is at least one plasticizing agent that imparts flexibility to the polymerized monomer formed on the wound, incision, or abrasion. The plasticizing agent preferably contains little or no moisture and should not significantly affect the polymerization of the monomer.

Examples of suitable plasticizers include acetyl tributyl citrate, dimethyl sebacate, triethyl phosphate, tri(2-ethylhexyl)phosphate, tri(p-cresyl) phosphate, glyceryl triacetate, glyceryl tributyrate, diethyl sebacate, dioctyl adipate, isopropyl myristate, butyl stearate, lauric acid, trioctyl trimellitate, dioctyl glutarate and mixtures thereof. Preferred plasticizers are tributyl citrate and acetyl tributyl citrate. In embodiments, suitable plasticizers include polymeric plasticizers, such as polyethylene glycol (PEG) esters and capped PEG esters or ethers, polyester glutarates and polyester adipates.

When present, component C) is at least one stabilizing agent that inhibits polymerization. Such stabilizing agents may also include mixtures of anionic stabilizing agents and radical stabilizing agents.

Examples of suitable anionic stabilizing agents include, but are not limited to, sultones (e.g., α-chloro-α-hydroxy-o-toluenesulfonic acid-γ-sultone), sulfur dioxide, sulfuric acid, sulfonic acid, lactone, boron trifluoride, organic acids, such as acetic acid or phosphoric acid, alkyl sulfate, alkyl sulfite, 3-sulfolene, alkylsulfone, alkyl sulfoxide, mercaptan, and alkyl sulfide and mixtures thereof. Preferable anionic stabilizing agents are acidic stabilizing agents of organic acids such as acetic acid or phosphoric acid. In embodiments, the amount of sulfur dioxide stabilizer is less than 100 ppm, preferably 5-75 ppm, and more preferably from about 20-50 ppm. The amount of sultone and/or trifluoracetic acid is about 500-3000 ppm.

Examples of suitable radical stabilizing agents include hydroquinone, hydroquinone monomethyl ether, catechol, pyrogallol, benzoquinone, 2-hydroxybenzoquinone, p-methoxy phenol, t-butyl catechol, butylated hydroxy anisole (BHA), butylated hydroxy toluene, and t-butyl hydroquinone. In embodiments, the amount of BHA is about 1,000-5,000 ppm.

Suitable acidic stabilizing agents include those having aqueous pKa ionization constants ranging from −12 to 7, about −5 to about 7, preferably from about −3.5 to about 6. For example, suitable acidic stabilizing agents include: hydrogen sulfide (pKa 7.0), carbonic acid (pKa 6.4), triacetylmethane (pKa 5.9), acetic acid (pKa 4.8), benzoic acid (pKa 4.2), 2,4-dinitrophenol (pKa 4.0), formic acid (pKa 3.7), nitrous acid (pKa 3.3), hydrofluoric acid (pKa 3.2), chloroacetic acid (pKa 2.9), phosphoric acid (pKa 2.2), dichloroacetic acid (pKa 1.3), trichloroacetic acid (pKa 0.7), 2,4,6-trinitrophenol (picric acid) (pKa 0.3), trifluoroacetic acid (pKa 0.2), sulfuric acid (pKa −3.0), sulfurous acid, and mixtures thereof. In embodiments, the amount of trifluoroacetic acid is about 500-1,500 ppm. Combinations of the above stabilizers, such as sulfur dioxide and sulfuric acid, boron trifluoride and sulfuric acid, sulfur dioxide and chloroacetic acid, boron trifluoride and chloroacetic acid, sulfur dioxide and trifluoroacetic acid, and boron trifluoride and triflouroacetic acid can be used.

When adding the acidic stabilizing agents mentioned above to the adhesive composition, the addition of plasticizing agents in amounts ranging from about 0.5 wt. % to about 16 wt. %, preferably from about 3 wt. % to about 9 wt. %, and more preferably from about 5 wt. % to about 7 wt. % provides increased film strength (e.g., toughness) of the polymerized monomer over polymerized monomers having amounts of plasticizing agents and acidic stabilizing agents outside of the above ranges.

The concentration of the acidic stabilizing agents utilized may vary depending on the strength of the acid. For example, when using acetic acid, a concentration of 80-200 ppm (wt/wt), preferably 90-180 ppm (wt/wt), and more preferably 100-150 ppm (wt/wt) may be utilized. When using a stronger acid such as phosphoric acid, a concentration range of 20-80 ppm (wt/wt), preferably, 30-70 ppm (wt/wt) and more preferably 40-60 ppm (wt/wt) may be utilized. In embodiments, the amount of trifluoroacetic acid is about 100 to 3000 ppm, preferably 500-1500 ppm. In other embodiments, the amount of phosphoric acid is about 10-200 ppm, preferably about 50-150 ppm, and more preferably about 75-125 ppm.

The compositions of the present invention may also include at least one biocompatible agent effective to reduce active formaldehyde concentration levels produced during in vivo biodegradation of the polymer (also referred to herein as “formaldehyde concentration reducing agents”). Preferably, this component is a formaldehyde scavenger compound. Examples of formaldehyde scavenger compounds useful in this invention include sulfites; bisulfites; mixtures of sulfites and bisulfites; ammonium sulfite salts; amines; amides; imides; nitriles; carbamates; alcohols; mercaptans; proteins; mixtures of amines, amides, and proteins; active methylene compounds such as cyclic ketones and compounds having a b-dicarbonyl group; and heterocyclic ring compounds free of a carbonyl group and containing an NH group, with the ring made up of nitrogen or carbon atoms, the ring being unsaturated or, when fused to a phenyl group, being unsaturated or saturated, and the NH group being bonded to a carbon or a nitrogen atom, which atom is directly bonded by a double bond to another carbon or nitrogen atom.

Bisulfites and sulfites useful as the formaldehyde scavenger compound in this invention include alkali metal salts such as lithium, sodium and potassium salts, and ammonium salts, for example, sodium bisulfite, potassium bisulfite, lithium bisulfite, ammonium bisulfite, sodium sulfite, potassium sulfite, lithium sulfite, ammonium sulfite, and the like.

Examples of amines useful in this invention include the aliphatic and aromatic amines such as, for example, aniline, benzidine, aminopyrimidine, toluene-diamine, triethylenediamine, diphenylamine, diaminodiphenylamine, hydrazines and hydrazide.

Suitable proteins include collagen, gelatin, casein, soybean protein, vegetable protein, keratin and glue. The preferred protein for use in this invention is casein.

Suitable amides for use in this invention include urea, cyanamide, acrylamide, benzamide, and acetamide. Urea is a preferred amide.

Suitable alcohols include phenols, 1,4-butanediol, d-sorbitol, and polyvinyl alcohol.

Examples of suitable compounds having a b-dicarbonyl group include malonic acid, acetylacetone, ethylacetone, acetate, malonamide, diethylmalonate or another malonic ester.

Preferred cyclic ketones for use in this invention include cyclohexanone or cyclopentanone.

Examples of suitable heterocyclic compounds for use as the formaldehyde scavenger in this invention are disclosed, for example, in U.S. Pat. No. 4,127,382 (Perry) which is hereby incorporated by reference herein. Such heterocyclic compounds include, for example, benzimidazole, 5-methyl benzimidazole, 2-methylbenzimidazole, indole, pyrrole, 1,2,4-triazole, indoline, benzotriazole, indoline, and the like.

A preferred formaldehyde scavenger for use in this invention is sodium bisulfite.

In practicing the present invention, the formaldehyde concentration reducing agent, e.g., formaldehyde scavenger compound, is added in an effective amount to the cyanoacrylate. The “effective amount” is that amount sufficient to reduce the amount of formaldehyde generated during subsequent in vivo biodegradation of the polymerized cyanoacrylate. This amount will depend on the type of active formaldehyde concentration reducing agent, and can be readily determined without undue experimentation by those skilled in the art.

The formaldehyde concentration reducing agent may be used in this invention in either free form or in microencapsulated form. Other compositions are exemplified by U.S. patent application Ser. No. 08/714,288, incorporated by reference herein in its entirety.

When microencapsulated, the formaldehyde concentration reducing agent is released from the microcapsule continuously over a period of time during the in vivo biodegradation of the cyanoacrylate polymer.

For purposes of this invention, the microencapsulated form of the formaldehyde concentration reducing agent is preferred because this embodiment prevents or substantially reduces polymerization of the cyanoacrylate monomer by the formaldehyde concentration reducing agent, which increases shelf-life and facilitates handling of the monomer composition during use.

Microencapsulation of the formaldehyde scavenger can be achieved by many known microencapsulation techniques. For example, microencapsulation can be carried out by dissolving a coating polymer in a volatile solvent, e.g., methylene chloride, to a polymer concentration of about 6% by weight; adding a formaldehyde scavenger compound in particulate form to the coating polymer/solvent solution under agitation to yield a scavenger concentration of 18% by weight; slowly adding a surfactant-containing mineral oil solution to the polymer solution under rapid agitation; allowing the volatile solvent to evaporate under agitation; removing the agitator; separating the solids from the mineral oil; and washing and drying the microparticles. The size of the microparticles will range from about 0.001 to about 1000 microns.

The coating polymer for microencapsulating the formaldehyde concentration reducing agent should be polymers which undergo in vivo bioerosion, preferably at rates similar to or greater than the cyanoacrylate polymer formed by the monomer, and should have low inherent moisture content. Such bioerosion can occur as a result of the physical or chemical breakdown of the encapsulating material, for example, by the encapsulating material passing from solid to solute in the presence of body fluids, or by biodegradation of the encapsulating material by agents present in the body.

Examples of coating materials which can be used to microencapsulate the formaldehyde concentration reducing agent include polyesters, such as polyglycolic acid, polylactic acid, poly-1,4-dioxa-2-one, polyoxaltes, polycarbonates, copolymers of polyglycolic acid and polylactic acid, polycaprolactone, poly-b-hydroxybutyrate, copolymers of epsilon-caprolactone and delta-valerolactone, copolymers of epsilon-caprolactone and DL-dilactide, and polyester hydrogels; polyvinylpyrrolidone; polyamides; gelatin; albumin; proteins; collagen; poly(orthoesters); poly(anhydrides); poly(alkyl-2-cyanoacrylates); poly(dihyfropyrans); poly(acetals); poly(phophazenes); poly(urethanes); poly(dioxinones); cellulose; and starches.

Examples of the surfactant which can be added to the mineral oil include those commercially available under the designations Triton x-100™ (octoxynol from Rohm & Haas), Tween 20™ (polysorbate 20 from ICI Americas) and Tween 80™ (polysorbate 80 from ICI Americas).

When present, component D) is a thickening agent. Suitable thickeners include, for example, polycyanoacrylates, polylactic acid, poly-1,4-dioxa-2-one, polyoxalates, polyglycolic acid, lactic-glycolic acid copolymers, polycaprolactone, lactic acid-caprolactone copolymers, poly-3-hydroxybutyric acid, polyorthoesters, polyalkyl acrylates, copolymers of alkylacrylate and vinyl acetate, polyalkyl methacrylates, and copolymers of alkyl methacrylates and butadiene. Examples of alkyl methacrylates and acrylates are poly(2-ethylhexyl methacrylate) and poly(2-ethylhexyl acrylate), also poly(butylmethacrylate) and poly(butylacrylate), also copolymers of various acrylate and methacrylate monomers, such as poly(butylmethacrylate-co-methylacrylate).

To improve the cohesive strength of adhesives formed from the compositions of this invention, difunctional monomeric cross-linking agents may be added to the monomer compositions of this invention. Such crosslinking agents are known. U.S. Pat. No. 3,940,362 to Overhults, which is hereby incorporated by reference, discloses such cross-linking agents. Examples of suitable crosslinking agents include alkyl bis(2-cyanoacrylates), triallyl isocyanurates, alkylene diacrylates, alkylene dimethacrylates, trimethylol propane triacrylate, and alkyl bis(2-cyanoacrylates). A catalytic amount of an amine activated free radical initiator or rate modifier may be added to initiate polymerization or to modify the rate of polymerization of the cyanoacrylate monomer/crosslinking agent blend.

The compositions of this invention may further contain fibrous reinforcement and colorants, i.e., dyes and pigments. Examples of suitable fibrous reinforcement include PGA microfibrils, collagen microfibrils, cellulosic microfibrils, and olefinic microfibrils. Examples of suitable colorants include 1-hydroxy-4-[4-methylphenyl-amino]-9,10 anthracenedione (D+C violet No. 2); disodium salt of 6-hydroxy-5-[(4-sulfophenyl)axo]-2-naphthalene-sulfonic acid (FD+C Yellow No. 6); 9-(o-carboxyphenyl)-6-hydroxy-2,4,5,7-tetraiodo-3H-xanthen-3-one, disodium salt, monohydrate (FD+C Red No. 3); 2-(1,3-dihydro-3-oxo-5-sulfo-2H-indol-2-ylidene)-2,3-dihydro-3-oxo-1H-indole-5-sulfonic acid disodium salt (FD+C Blue No. 2); and [phthalocyaninato (2-)] copper.

Other compositions that are contemplated by the present invention are exemplified by U.S. Pat. Nos. 5,624,669; 5,582,834; 5,575,997; 5,514,371; 5,514,372; and 5,259,835; the disclosures of all of which are hereby incorporated in their entirety by reference.

Compositions, including the polymerization initiators, rate modifiers, bioactive materials and/or flavorant, employed in the invention are preferably sterilizable such as by dry heat (e.g. above 100° C.), electron beam, gamma irradiation, ethylene oxide or hydrogen peroxide vapor, and other methods.

While the invention has been described with reference to preferred embodiments, the invention is not limited to the specific examples given, and other embodiments and modifications can be made by those skilled in the art without departing from the spirit and scope of the invention.

1-144. (canceled)

145. A method of applying at least one agent selected from the group consisting of bioactive materials, flavorants, polymerization initiators, and polymerization rate modifiers to an applicator tip for a cyanoacrylate adhesive applicator, comprising:
dissolving or dispersing said agent in a low boiling point solvent to form a solution;
applying said solution to said applicator tip; and
drying said applicator tip;
wherein the low boiling point solvent comprises methanol, and
wherein said applicator tip comprises a porous, absorbent, or adsorbent material;
such that when cyanoacrylate is dispensed through the applicator tip (a) less heat is generated than if the applicator tip was prepared using acetone as the solvent and/or (b) the amount of time required for polymerization is longer than if the applicator tip was prepared using acetone as the solvent.

146. The method of claim 145, wherein the agent is dissolved in the low boiling point solvent.

147. The method of claim 145, wherein the agent is selected from the group consisting of polysorbate 20, polysorbate 80, poloxamers, tetrabutylammonium bromide, alkylbenzylalkonium chloride, stannous octoate (tin (II) 2-ethylhexanoate), sodium tetradecyl sulfate, and dodecyldimethyl(3-sulfopropyl)ammonium hydroxide.

148. The method of claim 145, wherein the agent is alkylbenzyldimethylammonium chloride with an alkyl containing 6-18 carbon atoms, its pure components, or mixtures thereof.

149. The method of claim 145, wherein the agent comprises at least one member selected from the group consisting of antimicrobials, steroids, anesthetics, antifungal agents, anti-inflammatory agents, antiviral agents, and antitumor agents.

150. The method of claim 145, wherein the agent comprises crystal violet.

151. The method of claim 145, wherein the agent comprises a mixture of (i) at least one member selected from the group consisting of polymerization initiators and polymerization rate modifiers, and (ii) at least one member selected from the group consisting of bioactive materials and flavorants.

152. The method of claim 145, wherein the agent comprises at least one compound that is both (i) at least one member selected from the group consisting of polymerization initiators and polymerization rate modifiers and (ii) a bioactive material.

153. The method of claim 145, wherein said solvent is methanol.

154. The method of claim 145, wherein said solvent further comprises a low boiling point ketone or alcohol other than methanol.

155. The method of claim 145, wherein said solvent further comprises acetone.

156. The method of claim 145, comprising applying said solution to a distal end of the applicator tip and forming a concentration gradient of said agent that decreases from said distal end of the applicator tip towards a center and a proximal end of the applicator tip.

157. The method of claim 145, wherein said solution is applied to said applicator tip by a process comprising:
combining said solution and said applicator tip in a vessel;
sealing said vessel;
applying one of a vacuum or pressure to said vessel to degas air trapped in said applicator tip; and
releasing said vacuum or pressure.

158. The method of claim 145, wherein said applicator tip comprises a porous polyurethane, polyolefin, polyester, or polyamide.

159. The method of claim 145, wherein said applicator tip comprises porous polyethylene.

160. The method of claim 145, wherein said applicator tip comprises polyurethane foam.

161. The method of claim 158, wherein said applicator tip has an average pore size of about 1 μm to about 500 μm.

162. An applicator tip made by the method of claim 145.

163. A method of applying at least one agent selected from the group consisting of bioactive materials, flavorants, polymerization initiators, and polymerization rate modifiers to an applicator tip for a polymerizable adhesive applicator, comprising:
dissolving, dispersing or suspending said agent in a liquid medium to form a suspension or solution;
combining said suspension or solution and said applicator tip in a vessel;
sealing said vessel;
applying one of a vacuum or pressure to said vessel to degas air trapped in said applicator tip;
releasing said vacuum or pressure; and
optionally drying said applicator tip.

164. The method of claim 163, further comprising forcing said applicator tip below a surface of said suspension or solution.
